(19) United States
(12) Patent Application Publication
(10) Pub. No.: US 2023/0181843 A1
(43) Pub. Date: Jun. 15, 2023

(54) HEMOSTATIC SPRAY

(71) Applicant: FINEMEDIX CO., LTD., Daegu (KR)

(72) Inventor: Seong Ho HONG, Daegu (KR)

(21) Appl. No.: 18/080,165

(22) Filed: Dec. 13, 2022

(30) Foreign Application Priority Data
Dec. 15, 2021 (KR) 10-2021-0179791

(51) Int. Cl. A61M 11/02; A61L 24/08; A61L 24/10

(52) U.S. Cl. CPC A61M 11/02; A61L 24/08; A61L 24/104; A61L 2400/04; A61L 2400/06; A61M 2202/064; A61M 2205/75

(57) ABSTRACT

Proposed is a hemostatic sprayer for stopping a flow of blood from a bleeding portion of the human body, the hemostatic sprayer including a powder casing inside which an accommodation space is formed and which includes inlet and outlet ports, hemostatic in the form of powders being pre-stored in the accommodation space, and the inlet and outlet ports connecting thereto, and a receptacle inside which gas is compressed and pre-stored and which has a supply port on one side, the supply port separably connecting to the inlet port, wherein the supply port is open as a result of pressing by the powder casing, wherein, through the open supply port, the gas is supplied with a preset pressure toward the inlet port and flows into the accommodation space through the inlet port, and wherein the gas flowing is discharged together with the hemostatic through the outlet port while scattering the hemostatic.

### CROSS REFERENCE TO RELATED APPLICATION

The present application claims priority to Korean Patent Application No. 10-2021-0179791, filed Dec. 15, 2021, the entire contents of which is incorporated herein for all purposes by this reference.

### BACKGROUND OF THE INVENTION

### Field of the Invention

The present disclosure relates to a hemostatic sprayer having a structure in which a powder casing in which biocompatible hemostatic in the forms of powders is stored and an aerosol-type receptacle are coupled to each other. The hemostatic sprayer with this structure is capable of causing gas present inside the receptacle to be discharged as a result of pressing by the powder casing and more easily spraying the hemostatic present inside the powder casing onto a bleeding portion of the human body as a result of discharging the gas.

### Background Art

Usually, in a hospital, a bleeding portion of a patient’s body may be located through endoscopic examination, and, for the purpose of stopping a flow of blood from the bleeding portion, a clip is used, or a medical treatment, such as a high-frequency medical procedure, is performed.

The clip has a structure similar to that of tongs in such a manner as to easily stop the flow of blood from the human tissue cut from the human body. Various configurations of the clip for stopping the flow of blood are publicly known. Examples of the configuration in the related art for stopping the flow of blood include Korean Patent Nos. 10-2058581 (Hemostatic Clip for Endoscopy) and 10-0832957 (Endo-therapy Product System and Cartridge Including Treatment Device).

However, in a case where the bleeding portion of the human body is wide, a problem with a clip-based medical procedure for stopping the flow of blood as in the related art is that the flow of blood is difficult to sufficiently stop and that the flow of blood cannot be completely stopped because a clip-ligated portion of the human body is not wide.

In the case of the high-frequency medical treatment, a bleeding portion of the human body is cauterized with high-frequency heat in such a manner as to easily stop the flow of blood from the human tissue, and thus bleeding is minimized. Various high-frequency-based configurations for stopping the flow of blood are publicly known. Examples of the high-frequency-based configuration in the related art for stopping the flow of blood include Korean Patent Nos. 10-1954705 (High-frequency Treatment Device for Endoscopic Spinal Surgery) and 10-1168711 (Bleeding after the Biopsy Device).

However, in the case where the bleeding portion of the human body is wide, a problem with the high-frequency-based medical procedure for stopping the flow of blood as in the related art is that it takes much time to heat the bleeding portion of the human body up to a temperature at which the effect of stopping the flow of blood is achieved. As a result, burns occur widely, thereby uselessly destroying the human tissue. Moreover, the necessary effect of stopping the flow of blood cannot be achieved.

Furthermore, another problem with the clip-based medical procedure and the high-frequency-based medical procedure for stopping the flow of blood as in the related art is that there is a high likelihood that bleeding will reoccur due to active bleeding and severe bleeding among types of bleeding that occur during the medical treatment.

Accordingly, a medical procedure for stopping the flow of blood is required that can achieve the more increased effect of stopping the flow of blood from the bleeding portion of the human body than the clip-based medical procedure and the high-frequency-based medical procedure for stopping the flow of blood.

### DESCRIPTION OF THE RELATED ART

### Document of Related Art

(Patent Document 1) Korean Patent No. 10-2058581
(Patent Document 2) Korean Patent No. 10-0832957
(Patent Document 3) Korean Patent No. 10-1954705 (Patent Document 4) Korean Patent No. 10-1168711

### SUMMARY OF THE INVENTION

An object of the present disclosure, which is contrived to solve the above-mentioned problems, is to provide a hemostatic sprayer having a structure in which a powder casing in which biocompatible hemostatic in the forms of powders that can reduce the likeliness of bleeding reoccurring from a bleeding portion of the human body is stored is coupled to an aerosol-type receptacle. The hemostatic sprayer with this structure is configured to cause gas present inside the receptacle to be discharged as a result of pressing by the powder casing and to discharge the gas together with the hemostatic to the outside as a result of discharging the gas. Accordingly, the hemostatic sprayer is capable of more easily spraying the hemostatic present inside the powder casing onto a bleeding portion of the human body, thereby increasing the effect of stopping a flow of blood from a bleeding portion of the human body.

The present disclosure is not limited to the above-mentioned object. From the following detailed description of the present disclosure, another object of the present disclosure that is not mentioned would be clearly understandable to a person of ordinary skill in the art to which the present disclosure pertains.

In order to accomplish the above-mentioned object, according to an aspect of the present disclosure, there is provided a hemostatic sprayer for stopping a flow of blood from a bleeding portion of the human body, the hemostatic sprayer including: a powder casing inside which an accommodation space is formed and which includes an inlet port and an outlet port, hemostatic in the form of powders being pre-stored in the accommodation space, and the inlet port and the outlet port connecting to the accommodation space; and a receptacle inside which gas is compressed and pre-stored and which has a supply port on one side, the supply port separably connecting to the inlet port in the powder casing, wherein the supply port in the receptacle is open as a result of pressing by the powder casing, wherein, through the open supply port, the pre-stored gas is supplied with preset pressure toward the inlet port and flows into the accommodation space through the inlet port, and wherein the gas flowing into the accommodation space is discharged together with the hemostatic through the outlet port while scattering the hemostatic present inside the accommodation space.

In the hemostatic sprayer, the gas compressed and pre-stored in the receptacle may be CO₂ gas.

In the hemostatic sprayer, the hemostatic may be chitosan or gelatin for forming a hemostatic membrane on a bleeding portion of the human body.

In hemostatic sprayer, the powder casing may further include a membrane filter that is interposed between the accommodation space and the inlet port and thus prevents the hemostatic from flowing into the receptacle through the supply port and prevents a foreign material from flowing into the accommodation space through the inlet port.

The hemostatic sprayer may further include a protective cap that is separably coupled to the inlet port before the inlet port in the powder casing and the supply port in the receptacle are connected to each other and that, while covering the inlet port, blocks a foreign material from flowing through the inlet port.

The hemostatic sprayer may further include a connection member configured to be coupled to the outlet port in the powder casing and possibly to connect the cannula that is to be inserted into the bleeding portion of the human body.

The hemostatic sprayer may further include an adjustment valve interposed between the outlet port and the connection member and adjusting an amount of the hemostatic to be discharged through the outlet port by being locked or unlocked.

The hemostatic sprayer may further include a cover casing accommodating and covering the powder casing and one side of the receptacle, thereby preventing the powder casing and the supply port in the receptacle from being damaged, the receptacle may further include a first coupling member that is at least one of a groove and a protrusion formed in and on the one side of the receptacle in such a manner as to have an arbitrary size, and the cover casing may further include a second coupling member formed on one side of the covering casing in such a manner that corresponds to the first coupling member and may be separably coupled to the first coupling member.

In the hemostatic sprayer, the cover casing may further include: a first guide hole formed in such a manner that a space in which an outside force for the pressing by the powder casing is exerted without any interference while one portion of the powder casing is exposed is provided even when the cover casing covers the powder casing and the one side of the receptacle; and a second guide hole formed in such a manner that the outlet port in the powder casing is exposed and that the hemostatic is discharged up to the outside through the outlet port.

The hemostatic sprayer according to the present disclosure facilitate an operation of stopping the flow of blood from the bleeding portion of the human body using the biocompatible hemostatic in the form of powders and thus improving an effect of stopping the flow of blood. Accordingly, the likelihood of bleeding reoccurring from the bleeding portion of the human body can be reduced more effectively than is the case with a clip-based medical procedure and a high frequency-based procedure for stopping the flow of blood in the related art.

In addition, according to the present disclosure, a structure for realizing a technique of facilitating hemostatic injection more easily may be employed through the configuration where the powder casing in which the hemostatic is stored is coupled to the receptacle, which is an aerosol type, where the gas present inside the receptacle is discharged as a result of the pressing by the powder casing, and where the hemostatic is discharged together to the outside as a result of discharging the gas. Moreover, through the configuration where the powder casing and the receptacle are easily separated and coupled according to a user’s need, the compact-type powder casing is freely portable when not in use and, at the same time, is replaceable in a more convenient manner when the hemostatic or the gas is used up. Thus, there is an advantage in that higher productivity and portability of the hemostatic sprayer can be achieved.

Moreover, according to the present disclosure, the connection member is configured to connect the cannula, and the adjustment valve is configured to adjust the amount of the hemostatic to be discharged. Thus, through this configuration, a more precise and efficient medical treatment of the bleeding portion of the human body can be performed.

Furthermore, according to the present disclosure, the membrane filter is configured to be provided on the powder casing, and the protective cap is configured to cover the inlet port in the powder casing in advance of coupling the powder casing and the receptacle to each other. Through this configuration, the structural reliability of the hemostatic sprayer can be improved. The durability of the hemostatic sprayer can be improved through the cover casing covering the powder casing and one side of the receptacle. Particularly, the cover casing is configured to have the first and second guide holes therein. Thus, a space for the pressing by the powder casing, the discharging of the hemostatic, and the connecting of the cannula can be provided when the cover casing covers the powder casing and one side of the receptacle.

However, the present disclosure is not limited to the above-mentioned object. From the following detailed description of the present disclosure, another effect of the present disclosure that can be achieved, but not mentioned, would be clearly understandable to a person of ordinary skill in the art to which the present disclosure pertains.

### BRIEF DESCRIPTION OF THE DRAWINGS

FIG. 1 is a perspective view schematically illustrating a hemostatic sprayer according to a first embodiment of the present disclosure.

FIG. 2 is a side view schematically illustrating the hemostatic sprayer according to the first embodiment of the present disclosure.

FIG. 3 is a front view schematically illustrating the hemostatic sprayer according to the first embodiment of the present disclosure.

FIG. 4 is a cross-sectional view illustrating the hemostatic sprayer according to the first embodiment of the present disclosure, taken along line A-A′ on FIG. 1.

FIG. 5 is a cross-sectional view illustrating a powder casing of a hemostatic sprayer according to a second embodiment of the present disclosure.

FIG. 6 is a view partially illustrating a receptacle of the hemostatic sprayer according to the second embodiment of the present disclosure.

FIG. 7 is a partial cross-sectional view illustrating a protective cap and a cover casing of the hemostatic sprayer according to the second embodiment of the present disclosure.

FIG. 8 is a partial cross-sectional view illustrating a state where a first coupling member of the receptacle and a second coupling member of the cover casing are coupled to each other.

### DETAILED DESCRIPTION OF THE INVENTION

With reference to the accompanying drawings, an embodiment of the present disclosure will be described below in sufficient detail to enable a person of ordinary skill in the art to make and use the claimed disclosure without undue experimentation. However, the embodiment of the present disclosure is only provided to describe structural and functional features of the present disclosure. The claimed scope of the present disclosure should not be construed as being limited to the embodiment described in the present specification. That is, various modifications to the embodiment may be possible, and the embodiment may be implemented in various forms. Therefore, equivalents of the present disclosure that may realize the technical idea of the present disclosure should be understood as falling within the claimed scope of the present disclosure. In addition, a specific embodiment is not intended to achieve all advantageous effects of the present disclosure or to achieve only them. Therefore, the claimed scope of the present disclosure should not be understood as being limited to the object or advantageous effects mentioned above.

The terms used in the present specification should be understood as follows.

The terms “first,” “second,” and so forth are used to differentiate one constituent element from another and should not impose any limitation on the claimed scope of the present disclosure. For example, a first constituent element may be named a second constituent element. Likewise, the second constituent element may also be named the first constituent element. It should be understood that a constituent element, when referred to as “connected” to a different constituent element, may be directly connected to the different constituent element and may be connected to the different constituent element with a third constituent element in between. In contrast, it should be understood that a constituent element, when referred to as “directly connected” to a different constituent element, may be connected to the different constituent element without a third constituent element in between. The same is true for terms referring to a relationship among constituent elements. That is, the terms, such as “between” and “directly between,” or “adjacent to” and “directly adjacent to,” should also be construed in the same manner.

It should be understood that, unless otherwise stated clearly in context, a noun in singular form has the same meaning as when used in plural form. Moreover, it should be understood that the terms “include,” “have,” and the like are meant to indicate that an implemented feature, a number, a step, an operation, a constituent element, a component, or a combination thereof is present and not to preclude the possibility that one or more other features, numbers, steps, operations, constituent elements, components, or combinations thereof will be present or added.

Unless otherwise defined, a term used in the present specification has the same meaning as usually understood by a person of ordinary skill in the art to which the present disclosure pertains. A term included in a dictionary that is usually used should be interpreted as having the same defined meaning in context as when used in the art. Unless otherwise stated clearly in the present specification, a term used in the present specification should not be interpreted as having a special or excessively literal meaning.

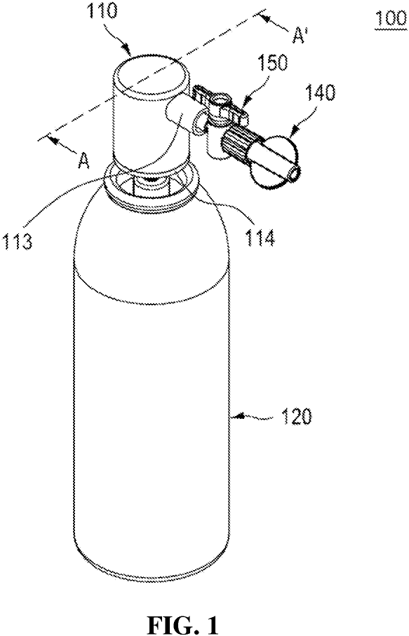

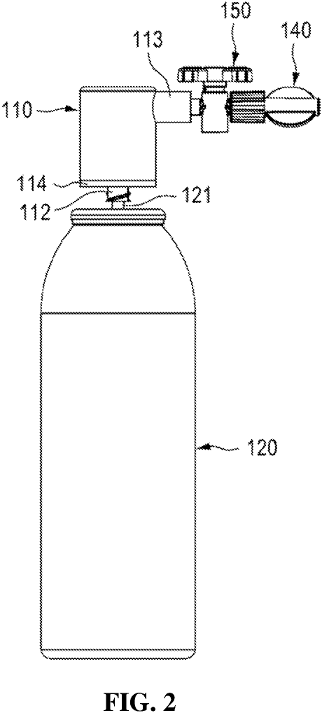

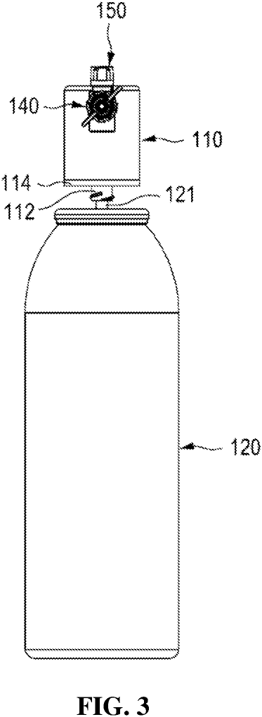

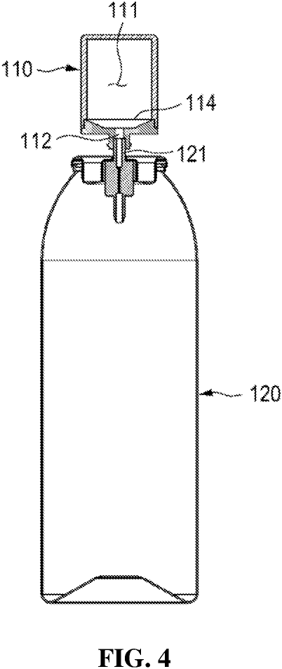

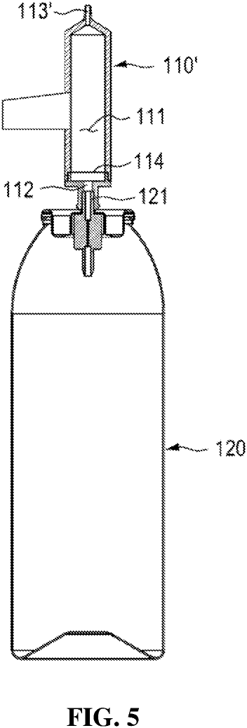

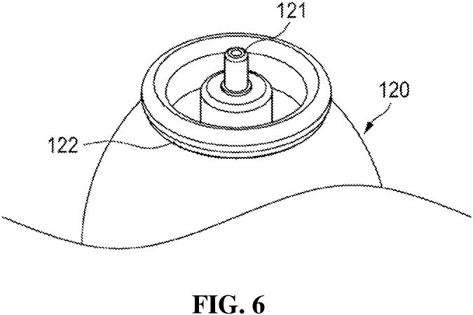

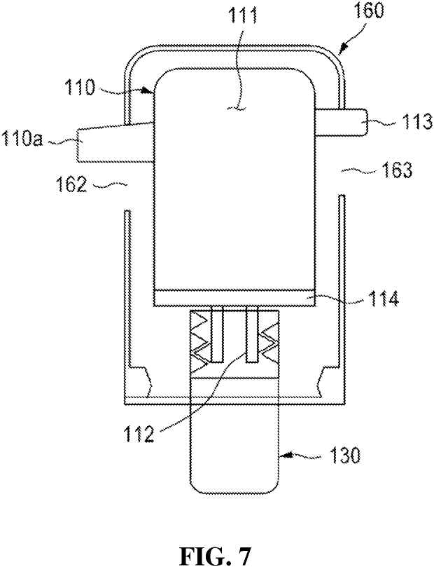

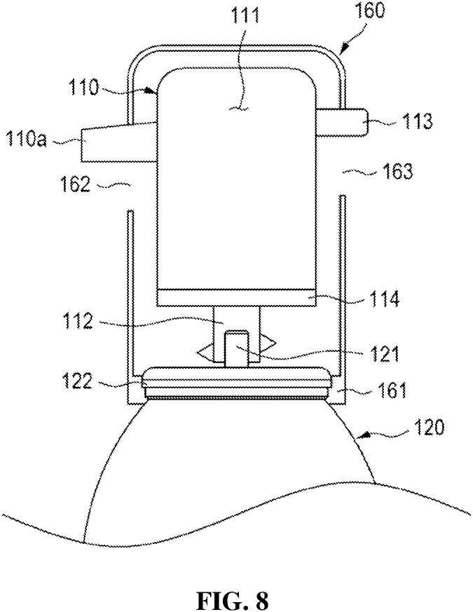

FIG. 1 is a perspective view schematically illustrating a hemostatic sprayer according to a first embodiment of the present disclosure. FIG. 2 is a side view schematically illustrating the hemostatic sprayer according to the first embodiment of the present disclosure. FIG. 3 is a front view schematically illustrating the hemostatic sprayer according to the first embodiment of the present disclosure. FIG. 4 is a cross-sectional view illustrating the hemostatic sprayer according to the first embodiment of the present disclosure, taken along line A-A′ on FIG. 1. FIG. 5 is a cross-sectional view illustrating a powder casing of a hemostatic sprayer according to a second embodiment of the present disclosure. FIG. 6 is a view partially illustrating a receptacle of the hemostatic sprayer according to the second embodiment of the present disclosure. FIG. 7 is a partial cross-sectional view illustrating a protective cap and a cover casing of the hemostatic sprayer according to the second embodiment of the present disclosure. FIG. 8 is a partial cross-sectional view illustrating a state where a first coupling member of the receptacle and a second coupling member of the cover casing are coupled to each other.

A hemostatic sprayer 100 according to the present disclosure will be described in detail below with reference to the accompanying drawings.

As illustrated in FIGS. 1 to 8, in order to stop a flow of blood from a bleeding portion of the human body by spraying biocompatible hemostatic onto the bleeding portion of the human body, the hemostatic sprayer 100 according to the present disclosure may be configured to include a powder casing 110, a receptacle 120, a protective cap 130, a connection member 140, an adjustment valve 150, and a cover casing 160.

However, the hemostatic sprayer 100 is not limited to the above-mentioned constituent elements. The hemostatic sprayer 100 may omit one or more constituent elements and, according to need, may further include a constituent element (for example, a catheter, or the like).

The powder casing 110 is configured to be coupled to the receptacle 120 described below, to press the receptacle 120 by means of an external force, and to inject the pre-stored biocompatible hemostatic by means of gas discharged from the receptacle 120 as a result of the pressing by the powder casing 110. According to the present disclosure, it is preferred that an accommodation space 111 is formed inside the powder casing 110 in such a manner that the hemostatic in the form of powders is pre-stored.

In addition, the powder casing 110 may include an inlet port 112 and an outlet port 113 that are connected to the above-mentioned accommodation space 111.

The inlet port 112 and the outlet port 113 may be provided on different sides, respectively, of the powder casing 110. Alternatively, as illustrated in FIG. 5, a powder casing 110′ of the hemostatic sprayer according to the second embodiment of the present disclosure may be configured in such a manner that the centers of the inlet port 112 and an outlet port 113′ may lie on a straight line. However, in order for the hemostatic to be smoothly scattered due to interference of the flow of the hemostatic by an inside wall of the powder casing 110′, it is more preferred that the powder casing 110 according to the first embodiment of the present disclosure is configured in such a manner that the inlet port 112 and the outlet port 113, as illustrated in figures other than FIG. 5, have the shape of the Korean letter “┐”

At this point, the degree to which the hemostatic is scattered may have an influence on an amount of remaining hemostatic and therefore is an important factor. Functions of the inlet port 112 and the outlet port 113 will be described in more detail in terms of operation in conjunction with a supply port 121, described below, in the receptacle 120.

According to the present disclosure, the hemostatic may be chitosan or gelatin in order to form a hemostatic membrane when the hemostatic is sprayed onto the bleeding portion of the human body, but is not limited thereto.

In addition, the hemostatic may be chitosan-catechol that results from a —OH group contained in a catechol group being partially oxidized to a = 0 group, then being oxidized by 1 to 20% on a catechol group mol basis through a dehydration condensation reaction with a — NH₂ group contained in chitosan, and thus being crosslinked.

Moreover, in order to form the hemostatic membrane when the hemostatic is sprayed onto the bleeding portion of the human body, the hemostatic may be polyamine or polaxamer to a terminal of which a thiol group is bonded.

Furthermore, the hemostatic may be one of the above-described compositions and may result from bonding the above-described compositions to each other.

The powder casing 110 may further include a membrane filter 114 that is interposed between the accommodation space 111 and the inlet port 112.

The membrane filter 114 performs a function of preventing the hemostatic from flowing into the receptacle 120 through the supply port 121 and preventing a foreign material from flowing into the accommodation space 111 through the inlet port 112.

The receptacle 120 compresses gas and pre-stores the compressed gas inside itself. The receptacle 120 may include the supply port 121 on one side thereof that separably connects to the inlet port 112 in the powder casing 110.

In this case, according to the present disclosure, an aerosol-type receptacle may be used as the receptacle 120. It is preferred that gas compressed and stored in the receptacle 120 is CO₂ gas. However, the receptacle 120 is not limited thereto.

The supply port 121 has a function of being open as a result of the pressing by the powder casing 110. When the supply port 121 is open, the pre-stored gas is supplied with a preset pressure toward the inlet port 112. Then, the pre-stored gas flows into the accommodation space 111 through the inlet port 112. The pre-stored gas flowing thereinto scatters the hemostatic present inside the accommodation space 111 and is discharged together with the hemostatic through the outlet port 113.

Configurations of the above-described receptacle 120 and supply port 121 and operations thereof in conjunction with the powder casing 110 are the same as or similar to configurations and operations of various known butane cans or sprayers that are regarded as being of an aerosol type. Therefore, more detailed descriptions thereof are omitted.

The protective cap 130 is a cap that closes the inlet port 112 in the powder casing 110 when the powder casing 110 and the receptacle 120 are separated from each other. The protective cap 130 is separably coupled to the inlet port 112 before the inlet port 112 in the powder casing 110 and the supply port 121 in the receptacle 120 are connected to each other. Thus, the protective cap 130 performs a function of blocking a foreign material from flowing through the inlet port 112 while covering the inlet port 112.

The connection member 140 is a connector provided to use a cannula. According to the present disclosure, it is preferred that the connection member 140 is configured to be coupled to the outlet port 113 in the powder casing 110 and possibly to connect the cannula that is to be inserted into the bleeding portion of the human body.

Various known connection ports may be used as the connection member 140. The connection member 140 is not limited to a structure thereof illustrated in the drawings. The connection member may be design-changed variously within the technical scope of the present disclosure by a person of ordinary skill in the art to which the present disclosure pertains.

The cannula and a catheter not illustrated may be connected together. In this case, the cannula is used for the purpose of causing the hemostatic to flow to the catheter. The catheter may inject the hemostatic directly toward the bleeding portion of the human body.

The adjustment valve 150 is a valve that adjusts an amount of the hemostatic to be discharged through the outlet port 113. According to the present disclosure, it is preferred that the adjustment valve 150 is interposed between the outlet port 113 and the connection member 140.

The adjustment valve 150 here may be at least one of 1-way, 2-way, 3-way valves that are capable of adjusting an amount of the hemostatic to be injected by being locked or unlocked.

The cover casing 160 is configured to prevent the powder casing 110 and the supply port 121 in the receptacle 120 from being damaged. It is preferred that the cover casing 160 accommodates and covers the powder casing 110 and one side of the receptacle 120.

According to the preferred embodiments of the present disclosure, it is preferred that the cover casing 160 is configured to be fixed to the receptacle 120. To this end, the receptacle 120 may further include a first coupling member 122 that is at least one of a groove and a protrusion formed in and on the one side of the receptacle 120 in which the supply port 121 is formed, in such a manner as to have an arbitrary size. The cover casing 160 may further include a second coupling member 161 that is formed in or on one side thereof in a manner that corresponds to the first coupling member 122, and is separably coupled to the first coupling member 122.

Furthermore, the cover casing 160 may be configured to further include a first guide hole 162 and a second guide hole 163 that are for providing a pressing space, a discharging space, and a connection space even when covering the powder casing 110 and the receptacle 120.

The first guide hole 162 is formed in one side of the cover casing 160 in such a manner as to provide a space in which an outside force for the pressing by the powder casing 110 is exerted without any interference while one portion of the powder casing 110 is exposed.

The second guide hole 163 is formed in the other side of the cover casing 160 in such a manner that the outlet port 113 in the powder casing 110 is exposed. Thus, the second guide hole 163 performs a function of discharging the hemostatic up to the outside through the outlet port 113.

Therefore, using the biocompatible hemostatic in the form of powders, the hemostatic sprayer 100 according to the present disclosure facilitates an operation of stopping the flow of blood from the bleeding portion of the human body and thus improving an effect of stopping the flow of blood. Accordingly, the likelihood of bleeding reoccurring from the bleeding portion of the human body can be reduced more than is the case with a clip-based medical procedure and a high frequency-based medical procedure for stopping the flow of blood in the related art.

In addition, a structure for realizing a technique of facilitating hemostatic injection more easily may be employed through the configuration where the powder casing 110 in which the hemostatic is stored is coupled to the receptacle 120, which is an aerosol type, where the gas present inside the receptacle 120 is discharged as a result of the pressing by the powder casing 110, and where the hemostatic is discharged together to the outside as a result of discharging the gas. Moreover, through the configuration where the powder casing 110 and the receptacle 120 are easily separated and coupled according to a user’s need, the compact-type powder casing is freely portable when not in use and, at the same time, is replaceable in a more convenient manner when the hemostatic or the gas is used up. Thus, higher productivity and portability of the hemostatic sprayer can be achieved.

Moreover, there is an advantage in that a more precise and efficient medical treatment of the bleeding portion of the human body can be performed through the configuration of the connection member 140 for connecting the cannula and the configuration of the adjustment valve 150 for adjusting the amount of the hemostatic to be discharged.

Furthermore, the structural reliability of the hemostatic sprayer can be improved through the configuration of the membrane filter 114 provided in the powder casing 110 and through the configuration of the protective cap 130 that covers the inlet port 112 in the powder casing 110 in advance of coupling the powder casing 110 and the receptacle 120 to each other. Moreover, the durability of the hemostatic sprayer can be improved through the cover casing 160 covering the powder casing 110 and one side of the receptacle 120. Particularly, the cover casing 160 is configured to have the first and second guide holes 161 and 162 therein. Thus, the effect of possibly providing the space for the pressing by the powder casing 110, the discharging of the hemostatic, and the connecting of the cannula can be achieved when the cover casing 160 covers the powder casing 110 and the receptacle 120.

The preferred embodiments of the present disclosure that are disclosed as described above are provided in sufficient detail to enable a person of ordinary skill in the art to make and use the present disclosure. The preferred embodiments of the present disclosure are described above. However, it would be understandable by a person of ordinary skill in the art that various modifications and alterations to the preferred embodiments of the present disclosure may be possibly made within the scope that does not depart from the claimed scope of the present disclosure. For example, a person of ordinary skill in the art may use combinations of the constituent elements according to the above-described preferred embodiments of the present disclosure. Therefore, the present disclosure is not limited to the preferred embodiments of the present disclosure that are described above and encompasses within the broadest claimed scope the principles and novel features of the present disclosure that are described above.

The present disclosure may be embodied in different specific forms within the scope that does not depart from the nature and gist of the present disclosure and from the essential features thereof. Therefore, the constituent elements that are in detail described above should be interpreted as being exemplary in all aspects, but not in a limited manner. The claimed scope of the present disclosure should be determined by proper construction of the following claims. All modifications to the preferred embodiments of the present disclosure fall equivalently within the claimed scope of the present disclosure. The present disclosure is not limited to the preferred embodiment described above and encompasses within the broadest claimed scope the principles and novel features of the present disclosure that are described above. In addition, an embodiment may be derived from combining the claims, and a new claim to the embodiment may be included in an amendment after the application is filed.

What is claimed is:

1. A hemostatic sprayer for stopping a flow of blood from a bleeding portion of the human body, the hemostatic sprayer comprising:
a powder casing having an accommodation space formed therein to store hemostatic in the form of powders and comprising an inlet port and an outlet port respectively connecting to the accommodation space; and
a receptacle configured to store compressed gas and having a supply port on one side thereof, the supply port separably connecting to the inlet port of the powder casing,
wherein the supply port of the receptacle is open due to a pressing by the powder casing,
wherein, through the open supply port, the stored compressed gas is supplied with a preset pressure toward the inlet port and flows into the accommodation space through the inlet port, and
wherein the gas flowing into the accommodation space is discharged together with the hemostatic through the outlet port while scattering the hemostatic present inside the accommodation space.

2. The hemostatic sprayer of claim 1, wherein the compressed gas stored in the receptacle is CO₂ gas.

3. The hemostatic sprayer of claim 1, wherein the hemostatic is chitosan or gelatin for forming a hemostatic membrane on the bleeding portion of the human body.

4. The hemostatic sprayer of claim 1, wherein the powder casing further comprises a membrane filter that is interposed between the accommodation space and the inlet port to prevent the hemostatic from flowing into the receptacle through the supply port and prevent a foreign material from flowing into the accommodation space through the inlet port.

5. The hemostatic sprayer of claim 1, further comprising:
a protective cap that is separably coupled to the inlet port before the inlet port in the powder casing and the supply port in the receptacle are connected to each other and that, while covering the inlet port, blocks a foreign material from flowing through the inlet port.

6. The hemostatic sprayer of claim 1, further comprising:
a connection member configured to be coupled to the outlet port in the powder casing to connect a cannula that is to be inserted into the bleeding portion of the human body.

7. The hemostatic sprayer of claim 6, further comprising:
an adjustment valve interposed between the outlet port and the connection member and adjusting an amount of the hemostatic to be discharged through the outlet port by being locked or unlocked.

8. The hemostatic sprayer of claim 1, further comprising:
a cover casing accommodating and covering the powder casing and one side of the receptacle, thereby preventing the powder casing and the supply port in the receptacle from being damaged,
wherein the receptacle further comprises:
a first coupling member that is at least one of a groove and a protrusion formed in and on the one side of the receptacle to have an arbitrary size, and
wherein the cover casing further comprises:
a second coupling member formed on one side of the cover casing to correspond to the first coupling member and is separably coupled to the first coupling member.

9. The hemostatic sprayer of claim 8, wherein the cover casing further comprises:
a first guide hole formed to provide a space in which an outside force for the pressing by the powder casing is exerted without any interference while one portion of the powder casing is exposed even when the cover casing covers the powder casing and the one side of the receptacle; and
a second guide hole formed in such a manner that the outlet port in the powder casing is exposed and that the hemostatic is discharged up to the outside through the outlet port.
